(19) United States
(12) Patent Application Publication
(10) Pub. No.: US 2025/0387385 A1
(43) Pub. Date: Dec. 25, 2025

(54) TREATMENT FOR GLOMERULAR DISEASES

(71) Applicant: ZYDUS Lifesciences Limited, Gurjarat (IN)

(72) Inventors: Mukul JAIN, Gujarat (IN); Amit Arvind JOHARAPURKAR, Gujarat (IN); Vishal Jagjivanbhai PATEL, Gujarat (IN)

(73) Assignee: ZYDUS Lifesciences Limited, Gurjarat (IN)

(21) Appl. No.: 18/878,500

(22) PCT Filed: Jun. 23, 2023
(86) PCT No.: PCT/IB2023/056501

§ 371 Date: Dec. 23, 2024

(30) Foreign Application Priority Data
Jun. 24, 2022 (IN) 202221036303

(51) Int. Cl. A61K 31/4704; A61K 31/454; A61K 31/506; A61P 13/12

(52) U.S. Cl. CPC A61K 31/4704; A61K 31/454; A61K 31/506; A61P 13/12

(57) ABSTRACT

The present invention relates to the development of therapeutic compound for the treatment of glomerular diseases. Specifically, present invention relates to use of compound of formula (Ia) or its pharmaceutically acceptable salt or combination thereof or pharmaceutical composition thereof for the treatment of glomerular diseases.

### FIELD OF THE INVENTION

Present invention relates to the development of therapeutic compound for the treatment of glomerular diseases. Specifically, present invention relates to use of compound of formula (Ia) or its pharmaceutically acceptable salt or combination thereof or pharmaceutical composition thereof for the treatment of glomerular diseases.

### BACKGROUND OF THE INVENTION

Glomeruli is the functional part of nephron, that is involved in the filtration and clearing the waste material from the body. The diseases affecting the glomeruli is known as glomerulopathy. It may be of inflammatory or non-inflammatory origin. The impairment of the function of glomeruli deteriorates the function of kidney. The glomerulopathy is also associated with hematuria or proteinuria, dysfunction of the endothelium, glomerular filtration barrier or podocyte. Subsequently, leads to proteinuria, hypoalbuminemia, edema, and hyperlipidemia known as nephrotic/nephritic syndrome. The glomerulopathy can also be of genetic origin such as IgAN, IgMAN, C3G, aHUS, iMN, Alport syndrome, autosomal dominant polycystic kidney disease and LN. Other diseases associated or enhances the inflammatory nephropathy is lupus nephritis if caused by systemic lupus erythematosus. Glomerulopathy develops or accelerated in presence of obesity, diabetes, and another comorbidity. Scarring of glomeruli or the blood vessels in glomeruli also affect the function of kidney, and is known as glomerulosclerosis. Focal segmental glomerulosclerosis (FSGS) or nodular glomerulosclerosis, are the forms of glomerulosclerosis. FSGC leads to end-stage renal disease, and is irreversible lead to nephrotic syndrome. Glomerulopathy or glomerulosclerosis can develop without any known cause or can also be secondary to the drugs, toxins or underlying disease. It is reported that chronic hypoxia leads to anemia, and is one of the complication and cause of the development of renal diseases such as glomerulopathy and glomerulosclerosis (1-3). Glomerulonephritis is also called glomerular disease. It is a type of kidney disease caused by damage to your glomeruli due to over activation of your immune system. This damage means the glomeruli cannot do their job to remove waste and fluid like they should.

Oxygen is an important factor which regulates acute and chronic inflammation. Oxygen levels in the tissues are sensed by hypoxia-inducible factors (HIFs: HIF-1 and HIF-2), regulated by prolyl hydroxylase enzymes (4). Activation of HIF prevents nephropathy and ischemia-reperfusion injury (5,6). Inhibition of PHD can stabilize HIF thus increasing the availability of HIF at the site of inflammation. Hypoxia inducible factor (HIF) regulates erythropoietin (EPO) secretion and inhibition of PHD thus increases EPO by stabilizing HIF. Thus, PHD inhibitor reduces anemia, and this increases oxygen to the kidney and progression of glomerulopathy or glomerulosclerosis may be delayed. HIF regulates inflammatory stimuli and mediators of inflammation (7,8). HIF has been reported to regulate nuclear factor-κB (NF-κB) and extracellular signal-regulated kinase (ERK) mediated inflammatory pathways (9). Desidustat is a PHD inhibitor currently approved for the treatment of chronic kidney disease-associated anemia in India. It is reported that Desidustat treatment stabilizes HIF and thus induces erythropoiesis in animal model of anemia (10). Desidustat also improves hemoglobin in clinical trials (11). Desidustat treatment reduced IL-6 and IL-1β levels in ischemia condition (12). These inflammatory markers were increased in renal dysfunction either nephropathy or nephritis (13). It also decreases SOD and MDA thus decreases oxidative stress (12). The standard therapy used glomerulopathy or glomerulonephritis are the steroid to suppress inflammation or anti-infective agents. Other agents such as RAAS inhibitors, mineralocorticoid antagonists, SGLT2 inhibitors, complement system inhibitors, anti-diabetic agents, anti-hyperlipidemic, diuretic or other agents used in the management of symptoms of glomerular disease or renal dysfunction or ESRD. Other investigations therapy may be useful in reducing progression or reversal of these diseases such as anti-inflammatory agents, NRF2 regulators, endothelin antagonist, immunomodulators, ACE inhibitors, discoidin domain receptor 1 inhibitors, osteopontin blocking agents, vasopressin receptor antagonists, gene editing therapy, or stem cell therapy. Thus, compound of formula (Ia) may be used to in the management of glomerulopathy or glomerulonephritis, and related diseases, and associated complications, either alone or in combination of agents mentioned above.

Some of the prolyl hydroxylase inhibitors have been disclosed in EP 661269, WO 2007070359, WO 2008076425, WO 2011007856, WO 2012106472, WO 2013043621, WO 2004108681 and WO 2008002576 covers the prolyl hydroxylase inhibitors. Pharmaceutical composition for treatment of oxidative stress disorders and treatment of hemoglobin disorders have been disclosed in WIPO publications WO 2014200773, WO 2017027810 and WO 2019028150 respectively.

WO 2014102818 discloses compounds of the following general formula

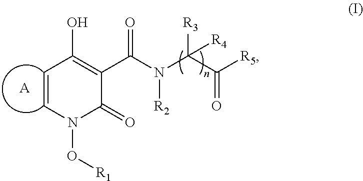

The compound of formula (Ia) as given below

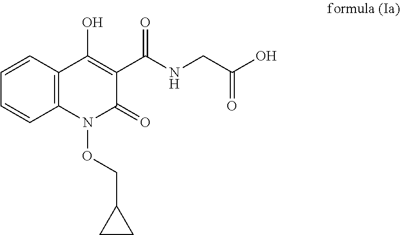 [SMILES: O=C(O)CNC(=O)c1c(O)c2ccccc2n(OCC2CC2)c1=O]

and its pharmaceutically acceptable salts are found effective in the treatment of glomerular diseases.

### OBJECTIVES OF THE INVENTION

In an embodiment, the present invention provides a method of treating glomerular diseases using compound of formula (Ia) or its pharmaceutically acceptable salts.

In another embodiment, the present invention provides a method of treating glomerular disease using combination of compound of formula (Ia) or its pharmaceutically acceptable salts with suitable second therapeutic agent selected from suitable Factor B inhibitors or suitable Angiotensin II receptor antagonist or suitable Factor D inhibitors or suitable C3 inhibitors or suitable C5 inhibitors or suitable C6 inhibitors or suitable Lectin pathway inhibitors or suitable Properdin inhibitors or suitable C9 antibody and multitarget complement inhibitor.

In another embodiment, the present invention provides a method of treating glomerular disease using combination of compound of formula (Ia) or its pharmaceutically acceptable salts with suitable Factor B inhibitors.

In another embodiment, the present invention provides a method of treating glomerular disease using combination of compound of formula (Ia) or its pharmaceutically acceptable salts with suitable Angiotensin II receptor antagonist.

In another embodiment, the present invention provides a suitable pharmaceutical composition comprising compound of formula (Ia) or its pharmaceutically acceptable salts for the treatment of glomerular diseases.

In another embodiment, the present invention provides a combination of compound of formula (Ia) or its pharmaceutically acceptable salts with suitable second therapeutic agent selected from suitable Factor B inhibitors or suitable Angiotensin II receptor antagonist or suitable Factor D inhibitors or suitable C3 inhibitors or suitable C5 inhibitors or suitable C6 inhibitors or suitable Lectin pathway inhibitors or suitable Properdin inhibitors or suitable C9 antibody and multitarget complement inhibitor.

In another embodiment, the present invention provides a combination of compound of formula (Ia) or its pharmaceutically acceptable salts with suitable Factor B inhibitors.

In another embodiment, the present invention provides a combination of compound of formula (Ia) or its pharmaceutically acceptable salts with suitable Angiotensin II receptor antagonist.

In another embodiment, the present invention provides a suitable pharmaceutical composition comprising compound of formula (Ia) or its pharmaceutically acceptable salts and suitable Factor B inhibitors for the treatment of glomerular diseases.

In another embodiment, the present invention provides a suitable pharmaceutical composition comprising compound of formula (Ia) or its pharmaceutically acceptable salts and suitable Angiotensin II receptor antagonist for the treatment of glomerular diseases.

In another embodiment, present invention provides use of compound of formula (I) or its pharmaceutically acceptable salts for the treatment of glomerular diseases.

In another embodiment, present invention provides use of combination of compound of formula (I) or its pharmaceutically acceptable salts with suitable Factor B inhibitors for the treatment of glomerular diseases.

In another embodiment, present invention provides use of combination of compound of formula (I) or its pharmaceutically acceptable salts with suitable Angiotensin II receptor antagonist for the treatment of glomerular diseases.

In an embodiment, compound of formula (Ia) or its pharmaceutically acceptable salts alone or suitable combination thereof for use may be further characterized according to a reduction in the amount of urine protein.

In another embodiment, the present invention provides the administration of compound of formula (Ia) or its pharmaceutically acceptable salts alone or in combination with suitable second therapeutic agents for the treatment of glomerular diseases.

In another embodiment, the present invention compound of formula (Ia) or its pharmaceutically acceptable salts or suitable combination thereof for the treatment of glomerular disease in patient with diabetes.

### BRIEF DESCRIPTION OF DRAWINGS

FIG. 1A. Effect of compound of formula (Ia) and its combination with Iptacopan on serum creatinine (A) in LPS treated mice;

FIG. 1B. Effect of compound of formula (Ia) and its combination with Iptacopan on serum urea (B) in LPS treated mice;

FIG. 1C. Effect of compound of formula (Ia) and its combination with Iptacopan on urine total protein (C) in doxorubicin treated mice;

FIG. 1D. Effect of compound of formula (Ia) and its combination with Fimasartan on urine total protein (D) in BSA overload induced mice.

FIG. 2A. Effect of compound of formula (Ia) on Urine albumin (A) in 5/6 nephrectomized rat;

FIG. 2B. Effect of compound of formula (Ia) on Serum creatinine (B) in 5/6 nephrectomized rat;

FIG. 2C. Effect of compound of formula (Ia) on Serum urea (C) in 5/6 nephrectomized rat;

FIG. 2D. Effect of compound of formula (Ia) on Urine albumin (D) in db/db mice;

FIG. 2E. Effect of compound of formula (Ia) on Serum creatinine (E) in db/db mice;

FIG. 2F. Effect of compound of formula (Ia) on Serum urea (F) in db/db mice.

### SUMMARY OF THE INVENTION

Present invention relates to compound of formula (Ia) or its pharmaceutically acceptable salts or combination thereof for the treatment of glomerular diseases. Invention also relates to pharmaceutical composition comprising compound of formula (Ia) or pharmaceutically acceptable excipients useful for the treatment of glomerular diseases.

### DESCRIPTION OF THE INVENTION

### Definition

The terms ‘treatment’ or ‘treat’ refer to slowing, stopping, or delaying the progression of the disease or clinical symptoms in a patient, as evidenced by a decrease or elimination of a clinical or diagnostic symptom of the disease, disorder or condition. The term ‘subject’ refer to a mammals. The term ‘effective amount’ in the context of the administration of the amount of the drug substance sufficient to have the desired effect. The term ‘pharmaceutically acceptable’ use embraces both human and veterinary use. A compound of formula (Ia) is Desidustat.

### Method of Treating Glomerular Disease by Compound of Formula (Ia)

In an embodiment the present invention provides a method of treating glomerular disease in a subject, comprising administering an effective amount of compound of formula (Ia) or its pharmaceutically acceptable salts; wherein formula (Ia) is represented by:

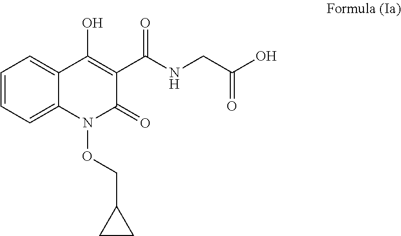 [SMILES: O=C(O)CNC(=O)c1c(O)c2ccccc2n(OCC2CC2)c1=O]

The glomerular disease includes Nephrotic Syndrome, Focal Segmental Glomerulosclerosis (FSGS), Minimal Change Disease (MCD), Membranous Nephropathy (MN), IgA Nephropathy (IgAN), C3 Glomerulopathy (MPGN3), Diabetic nephropathy, Lupus nephritis and other condition associated with Glomerular Diseases.

Pharmaceutically acceptable salts of the compound of formula (Ia) is selected from metal salt, amine base salt and amino acid salt.

Wherein metal salt is selected from calcium, sodium, potassium, lithium, barium, strontium, magnesium, cesium, copper, cobalt, iron, manganese, lead, aluminum, cadmium, silver, zinc, ammonium and the like; wherein amine base salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane-1,6-diamine, 2-(2-aminoethoxy) ethanamine, N-methylmorpholine, and N-ethylmorpholine; wherein amino acid salt is selected from alanine, lysine, arginine, histidine, threonine, proline, glutamine and glycine. In certain embodiments, compounds of formula (Ia) or its pharmaceutically acceptable salts, for administration to a subject at a dose in the range of 1 mg to 500 mg. In certain embodiments, the compound of formula (Ia) or a pharmaceutically acceptable salts for administration to a subject to provide the compound of formula (Ia) or a pharmaceutically acceptable salts at a dose of 1 mg to 150 mg. In certain embodiments, the compound of formula (Ia) or a pharmaceutically acceptable salts for administration to a subject to provide the compound of formula (Ia) or a pharmaceutically acceptable salts at a dose of 25 mg, 50 mg and 100 mg to the subject.

In a further embodiment, the compound of formula (Ia) or its pharmaceutically acceptable salts is administered to a subject in an amount of about 1 mg to about 500 mg, about 50 mg to about 450 mg, about 100 mg to about 400 mg, about 150 mg to about 350 mg, about 200 mg to about 300 mg, about 1 mg to about 50 mg, about 1 mg to about 25 mg to the subject.

In certain embodiments, compounds of formula (Ia) or its pharmaceutically acceptable salts can be further characterized according to the dose of compound administered to a subject, where subject is animal or human.

In a further embodiment, the present invention provides effective amount of compound of formula (Ia) or its pharmaceutically acceptable salt is administered to subject by oral, parenteral, intravenous or intramuscular route of administration.

Method of Treating Glomerular Disease in Combination of Compound of Formula (Ia) or its Pharmaceutically Acceptable Salts with Other Suitable Inhibitors

In an embodiment, the present invention provides a method of treating glomerular disease in a subject, comprising administering a combination of compound of formula (Ia) or its pharmaceutically acceptable salts with suitable second therapeutic agent selected from suitable Factor B inhibitors or suitable Angiotensin II receptor antagonist or suitable Factor D inhibitors or suitable C3 inhibitors or suitable C5 inhibitors or suitable C6 inhibitors or suitable Lectin pathway inhibitors or suitable Properdin inhibitors or suitable C9 antibody and multitarget complement inhibitor.

Factor B inhibitors is Iptacopan; Angiotensin II receptor antagonist is Fimasartan, Azilsartan, Candesartan, Eprosartan, Losartan, Olmesartan, Telmisartan, Valsartan; Factor D inhibitors is selected from Danicopan, ALXN2050, BCX9930; C3 inhibitors is selected from pegcitacoplan and AMY 201; C5 inhibitors is selected from eculizumab, vilobelimab, RA 101348, DF2593A, Tesidolumab, SOBI-002, Ravulizumab, Cemdsiran, ARC1905 and Avacopan; C6 inhibitors is CP 010; Lectin pathway inhibitors is Narsoplimab; Properdin inhibitors is NM9401; C9 antibody and multitarget complement inhibitor is MFHR1.

Method of Treating Glomerular Disease in Combination of Compound of Formula (Ia) or its Pharmaceutically Acceptable Salts with Suitable Factor B Inhibitors

In an embodiment, the present invention provides a method of treating glomerular disease in a subject, comprising administering a combination of compound of formula (Ia) or its pharmaceutically acceptable salts with suitable Factor B inhibitors.

The glomerular disease includes Nephrotic Syndrome, Focal Segmental Glomerulosclerosis (FSGS), Minimal Change Disease (MCD), Membranous Nephropathy (MN), IgA Nephropathy (IgAN), C3 Glomerulopathy (MPGN3), Diabetic nephropathy, Lupus nephritis and other condition associated with Glomerular Diseases.

Pharmaceutically acceptable salts of the compound of formula (Ia) is selected from metal salt, amine base salt and amino acid salt.

Wherein metal salt is selected from calcium, sodium, potassium, lithium, barium, strontium, magnesium, cesium, copper, cobalt, iron, manganese, lead, aluminum, cadmium, silver, zinc, ammonium and the like; wherein amine base salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane-1,6-diamine, 2-(2-aminoethoxy) ethanamine, N-methylmorpholine, and N-ethylmorpholine; wherein amino acid salt is selected from alanine, lysine, arginine, histidine, threonine, proline, glutamine and glycine. Factor B inhibitor use in combination with compound of formula (Ia) or its pharmaceutically acceptable salts is Iptacopan.

In certain embodiments, compounds of formula (Ia) or its pharmaceutically acceptable salts, for administration to a subject at a dose in the range of 1 mg to 500 mg. In certain embodiments, the compound of formula (Ia) or a pharmaceutically acceptable salts for administration to a subject to provide the compound of formula (Ia) or a pharmaceutically acceptable salts at a dose of 1 mg to 150 mg. In certain embodiments, the compound of formula (Ia) or a pharmaceutically acceptable salts for administration to a subject to provide the compound of formula (Ia) or a pharmaceutically acceptable salts at a dose of 25 mg, 50 mg and 100 mg to the subject.

In a further embodiment, the compound of formula (Ia) or its pharmaceutically acceptable salts is administered to a subject in an amount of about 1 mg to about 500 mg, about 50 mg to about 450 mg, about 100 mg to about 400 mg, about 150 mg to about 350 mg, about 200 mg to about 300 mg, about 1 mg to about 50 mg, about 1 mg to about 25 mg to the subject.

In a further embodiment, Iptacopan is administered to a subject in an amount of about 1 mg to about 500 mg, about 50 mg to about 450 mg, about 100 mg to about 400 mg, about 150 mg to about 350 mg, about 200 mg to about 300 mg. In a certain embodiment, Iptacopan is administered in an amount of 200 mg.

In certain embodiments, compounds of formula (Ia) or its pharmaceutically acceptable salts can be further characterized according to the dose of compound administered to a subject, where subject is animal or human.

In a further embodiment, the present invention provides effective amount of compound of formula (Ia) or its pharmaceutically acceptable salt is administered to subject by oral, parenteral, intravenous or intramuscular route of administration.

Method of Treating Glomerular Disease in Combination of Compound of Formula (Ia) or its Pharmaceutically Acceptable Salts with Suitable Angiotensin II Receptor Antagonist

In an embodiment, the present invention provides a method of treating glomerular disease in a subject, comprising administering a combination of compound of formula (Ia) or its pharmaceutically acceptable salts with suitable Angiotensin II receptor antagonist. The glomerular disease includes Nephrotic Syndrome, Focal Segmental Glomerulosclerosis (FSGS), Minimal Change Disease (MCD), Membranous Nephropathy (MN), IgA Nephropathy (IgAN), C3 Glomerulopathy (MPGN3), Diabetic nephropathy, Lupus nephritis and other condition associated with Glomerular Diseases.

Pharmaceutically acceptable salts of the compound of formula (Ia) is selected from metal salt, amine base salt and amino acid salt.

Wherein metal salt is selected from calcium, sodium, potassium, lithium, barium, strontium, magnesium, cesium, copper, cobalt, iron, manganese, lead, aluminum, cadmium, silver, zinc, ammonium and the like; wherein amine base salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane-1,6-diamine, 2-(2-aminoethoxy) ethanamine, N-methylmorpholine, and N-ethylmorpholine; wherein amino acid salt is selected from alanine, lysine, arginine, histidine, threonine, proline, glutamine and glycine. Angiotensin II receptor antagonist use in combination with compound of formula (Ia) or its pharmaceutically acceptable salts is Fimasartan, Azilsartan, Candesartan, Eprosartan, Losartan, Olmesartan, Telmisartan and Valsartan.

In certain embodiments, compounds of formula (Ia) or its pharmaceutically acceptable salts, for administration to a subject at a dose in the range of 1 mg to 500 mg. In certain embodiments, the compound of formula (Ia) or a pharmaceutically acceptable salts for administration to a subject to provide the compound of formula (Ia) or a pharmaceutically acceptable salts at a dose of 1 mg to 150 mg. In certain embodiments, the compound of formula (Ia) or a pharmaceutically acceptable salts for administration to a subject to provide the compound of formula (Ia) or a pharmaceutically acceptable salts at a dose of 25 mg, 50 mg and 100 mg to the subject.

In a further embodiment, the compound of formula (Ia) or its pharmaceutically acceptable salts is administered to a subject in an amount of about 1 mg to about 500 mg, about 50 mg to about 450 mg, about 100 mg to about 400 mg, about 150 mg to about 350 mg, about 200 mg to about 300 mg, about 1 mg to about 50 mg, about 1 mg to about 25 mg to the subject.

In a further embodiment, Fimasartan is administered to a subject in an amount of about 1 mg to about 500 mg, about 50 mg to about 450 mg, about 100 mg to about 400 mg, about 150 mg to about 350 mg, about 200 mg to about 300 mg. In a further embodiment, Azilsartan is administered to a subject in an amount of about 1 mg to about 500 mg. In a further embodiment, Candesartan is administered to a subject in an amount of about 1 mg to about 200 mg. In a further embodiment, Eprosartan is administered to a subject in an amount of about 1 mg to about 1000 mg. In a further embodiment, Losartan is administered to a subject in an amount of about 1 mg to about 500 mg. In a further embodiment, Olmesartan is administered to a subject in an amount of about 1 mg to about 200 mg. In a further embodiment, Telmisartan is administered to a subject in an amount of about 1 mg to about 500 mg. In a further embodiment, Valsartan is administered to a subject in an amount of about 1 mg to about 500 mg.

In a certain embodiment, Fimasartan is administered to a subject in an amount of about 60 mg and 120 mg. In a further embodiment, Azilsartan is administered to a subject in an amount of about 40 mg and 80 mg. In a further embodiment, Candesartan is administered to a subject in an amount of about 4 mg, 8 mg, 16 mg and 32 mg. In a further embodiment, Eprosartan is administered to a subject in an amount of about 400 mg and 600 mg. In a further embodiment, Losartan is administered to a subject in an amount of about 25 mg, 50 mg and 100 mg. In a further embodiment, Olmesartan is administered to a subject in an amount of about 5 mg, 20 mg and 40 mg. In a further embodiment, Telmisartan is administered to a subject in an amount of about 20 mg, 40 mg and 80 mg. In a further embodiment, Valsartan is administered to a subject in an amount of about 40 mg, 80 mg, 160 mg and 320 mg.

In certain embodiments, compounds of formula (Ia) or its pharmaceutically acceptable salts can be further characterized according to the dose of compound administered to a subject, where subject is animal or human.

In a further embodiment, the present invention provides effective amount of compound of formula (Ia) or its pharmaceutically acceptable salt is administered to subject by oral, parenteral, intravenous or intramuscular route of administration.

### Pharmaceutical Composition of Compound of Formula (Ia) or its Pharmaceutically Acceptable Salts for Use in Treating Glomerular Diseases

In an embodiment, present invention provides a pharmaceutical composition comprising compound of formula (Ia) or its pharmaceutically acceptable salts optionally with one or more pharmaceutically acceptable excipients for use in treating glomerular diseases.

The glomerular disease includes Nephrotic Syndrome, Focal Segmental Glomerulosclerosis (FSGS), Minimal Change Disease (MCD), Membranous Nephropathy (MN), IgA Nephropathy (IgAN), C3 Glomerulopathy (MPGN3), Diabetic nephropathy, Lupus nephritis and other condition associated with Glomerular Diseases.

Pharmaceutically acceptable salts of the compound of formula (Ia) is selected from metal salt, amine base salt and amino acid salt.

Wherein metal salt is selected from calcium, sodium, potassium, lithium, barium, strontium, magnesium, cesium, copper, cobalt, iron, manganese, lead, aluminum, cadmium, silver, zinc, ammonium and the like; wherein amine base salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane-1,6-diamine, 2-(2-aminoethoxy) ethanamine, N-methylmorpholine, and N-ethylmorpholine; wherein amino acid salt is selected from alanine, lysine, arginine, histidine, threonine, proline, glutamine and glycine. The pharmaceutically acceptable excipients are selected at least one from diluent, binders, disintegrating agents, lubricating agents, glidant agent, sweetener, pH modifier, suspending agent or viscosity modifying agent, flavouring agent and optionally coating redimix.

Diluents include, but are not limited to lactose monohydrate, lactose, microcrystalline cellulose, polymethacrylates selected from Eudragit, potassium chloride, sulfobutylether b-cyclodextrin, sodium chloride, spray dried lactose, and preferably sulfobutyl ether b-cyclodextrin combinations thereof and other such materials known to those of ordinary skill in the art.

Binders include, but are not limited to hypromellose 3 Cps, carbomers selected from carbopol, gellan, gum Arabic, hydrogenated vegetable oil, polymethacrylates selected from Eudragit, xanthan, lactose and Zein combinations thereof and other such materials known to those of ordinary skill in the art.

Disintegrating agents include, but are not limited to, croscarmellose Sodium, bicarbonate salt, chitin, gellan gum, polacrillin potassium and docusate Sodium combinations thereof and other such materials known to those of ordinary skill in the art.

Glidant agents include, but are not limited to, colloidal silica, calcium silicate, magnesium silicate, silicon hydrogel, cornstarch, talc, combinations thereof and other such materials known to those of ordinary skill in the art.

Lubricant agents include, but are not limited to, calcium stearate, magnesium stearate, mineral oil, stearic acid, zinc stearate, glycerin behenate, hydrogenated vegetable oil, sodium stearyl fumarate and myristic Acid suitable combinations thereof and other such materials known to those of ordinary skill in the art.

Suitable pH modifying agents which maintain the pH of the formulation according to the present invention include, but are not limited to Citric acid and other similar excipients and their suitable combinations and other materials known to those of ordinary skill in the art.

Suspending agents or viscosity agent according to the present invention include, but are not limited to microcrystalline cellulose and carboxymethylcellulose sodium (Avicel CL 611) and other similar excipients and their suitable combinations and other materials known to those of

| Strength - 5 mg/ml | Strength - 20 mg/ml |
|---|---|
| [symbol] | [symbol] |

[symbol]

indicates data missing or illegible when filed

ordinary skill in the art.

Sweetener is selected from Sucrose and all such materials known to those of ordinary skill in the art.

Flavouring agent is selected from cherry flavour, orange flavour, mango flavour and all such fruit flavour known to those of ordinary skill in the art.

Coating redimix is selected from Opadry Pink all such materials known to those of ordinary skill in the art.

In certain embodiments, Pharmaceutical composition of compounds of formula (Ia) or its pharmaceutically acceptable salts are as below;

| Ingredients | mg/5 ml | mg/5 ml |
|---|---|---|
| compounds of formula (Ia) | 25.00 | 100.00 |
| Sucrose | 3085.66 | 3010.66 |
| Citric Acid, Anhydrous USP | 10.00 | 10.00 |
| Microcrystalline Cellulose | 164.85 | 164.85 |
| and Carboxymethylcellulose Sodium (Avicel CL 611) | | |
| Xanthan Gum NF (Xantural 75) | 6.49 | 6.49 |
| Cherry Flavour | 8.00 | 8.00 |

The stable pharmaceutical composition according to the present invention may be in the form of tablet or capsule or a powder or a suspension in a liquid or an aerosol formulation or solutions, preferably in the form of tablet or capsule or suspension.

In certain embodiments, compounds of formula (Ia) or its pharmaceutically acceptable salts, for administration to a subject at a dose in the range of 1 mg to 500 mg. In certain embodiments, the compound of formula (Ia) or a pharmaceutically acceptable salts for administration to a subject to provide the compound of formula (Ia) or a pharmaceutically acceptable salts at a dose of 1 mg to 150 mg. In certain embodiments, the compound of formula (Ia) or a pharmaceutically acceptable salts for administration to a subject to provide the compound of formula (Ia) or a pharmaceutically acceptable salts at a dose of 25 mg, 50 mg and 100 mg to the subject.

In a further embodiment, the compound of formula (Ia) or its pharmaceutically acceptable salts is administered to a subject in an amount of about 1 mg to about 500 mg, about 50 mg to about 450 mg, about 100 mg to about 400 mg, about 150 mg to about 350 mg, about 200 mg to about 300 mg, about 1 mg to about 50 mg, about 1 mg to about 25 mg to the subject.

In certain embodiments, compounds of formula (Ia) or its pharmaceutically acceptable salts can be further characterized according to the dose of compound administered to a subject, where subject is animal or human.

In a further embodiment, the present invention provides effective amount of compound of formula (Ia) or its pharmaceutically acceptable salt is administered to subject by oral, parenteral, intravenous or intramuscular route of administration.

Combination of Compound of Formula (Ia) or its Pharmaceutically Acceptable Salts with Other Suitable Inhibitors

In an embodiment, present invention provides a combination of compound of formula (Ia) or its pharmaceutically acceptable salts with suitable second therapeutic agent selected from suitable Factor B inhibitors or suitable Angiotensin II receptor antagonist or suitable Factor D inhibitors or suitable C3 inhibitors or suitable C5 inhibitors or suitable C6 inhibitors or suitable Lectin pathway inhibitors or suitable Properdin inhibitors or suitable C9 antibody and multitarget complement inhibitor.

Factor B inhibitors is Iptacopan; Angiotensin II receptor antagonist is Fimasartan, Azilsartan, Candesartan, Eprosartan, Losartan, Olmesartan, Telmisartan, Valsartan; Factor D inhibitors is selected from Danicopan, ALXN2050, BCX9930; C3 inhibitors is selected from pegcitacoplan and AMY 201; C5 inhibitors is selected from eculizumab, vilobelimab, RA 101348, DF2593A, Tesidolumab, SOBI-002, Ravulizumab, Cemdsiran, ARC1905 and Avacopan; C6 inhibitors is CP 010; Lectin pathway inhibitors is Narsoplimab; Properdin inhibitors is NM9401; C9 antibody and multitarget complement inhibitor is MFHR1.

Combination of Compound of Formula (Ia) or its Pharmaceutically Acceptable Salts with Suitable Factor B Inhibitors

In an embodiment, present invention provides a combination of compound of formula (Ia) or its pharmaceutically acceptable salts with suitable Factor B inhibitors.

Factor B inhibitor use in combination with compound of formula (Ia) or its pharmaceutically acceptable salts is Iptacopan.

Combination of Compound of Formula (Ia) or its Pharmaceutically Acceptable Salts with Suitable Angiotensin II Receptor Antagonist

In an embodiment, present invention provides a combination of compound of formula (Ia) or its pharmaceutically acceptable salts with suitable Angiotensin II receptor antagonist.

Angiotensin II receptor antagonist use in combination with compound of formula (Ia) or its pharmaceutically acceptable salts is Fimasartan, Azilsartan, Candesartan, Eprosartan, Losartan, Olmesartan, Telmisartan and Valsartan.

### Pharmaceutical Composition of Compound of Formula (Ia) or its Pharmaceutically Acceptable Salts and Suitable Factor B Inhibitors for Use in Treating Glomerular Diseases.

In an embodiment, present invention provides a pharmaceutical composition comprising compound of formula (Ia) or its pharmaceutically acceptable salts and suitable Factor B inhibitors optionally with one or more pharmaceutically acceptable excipients for use in treating glomerular diseases.

The glomerular disease includes Nephrotic Syndrome, Focal Segmental Glomerulosclerosis (FSGS), Minimal Change Disease (MCD), Membranous Nephropathy (MN), IgA Nephropathy (IgAN), C3 Glomerulopathy (MPGN3), Diabetic nephropathy, Lupus nephritis and other condition associated with Glomerular Diseases.

Pharmaceutically acceptable salts of the compound of formula (Ia) is selected from metal salt, amine base salt and amino acid salt.

Wherein metal salt is selected from calcium, sodium, potassium, lithium, barium, strontium, magnesium, cesium, copper, cobalt, iron, manganese, lead, aluminum, cadmium, silver, zinc, ammonium and the like; wherein amine base salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane-1,6-diamine, 2-(2-aminoethoxy) ethanamine, N-methylmorpholine, and N-ethylmorpholine; wherein amino acid salt is selected from alanine, lysine, arginine, histidine, threonine, proline, glutamine and glycine. The Factor B inhibitor is Iptacopan.

The pharmaceutically acceptable excipients are selected at least one from diluent, binders, disintegrating agents, lubricating agents, glidant agent, sweetener, pH modifier, suspending agent or viscosity modifying agent, flavouring agent and optionally coating redimix.

Diluents include, but are not limited to lactose monohydrate, lactose, microcrystalline cellulose, polymethacrylates selected from Eudragit, potassium chloride, sulfobutylether b-cyclodextrin, sodium chloride, spray dried lactose, and preferably sulfobutyl ether b-cyclodextrin combinations thereof and other such materials known to those of ordinary skill in the art.

Binders include, but are not limited to hypromellose 3 Cps, carbomers selected from carbopol, gellan, gum Arabic, hydrogenated vegetable oil, polymethacrylates selected from Eudragit, xanthan, lactose and Zein combinations thereof and other such materials known to those of ordinary skill in the art.

Disintegrating agents include, but are not limited to, croscarmellose Sodium, bicarbonate salt, chitin, gellan gum, polacrillin potassium and docusate Sodium combinations thereof and other such materials known to those of ordinary skill in the art.

Glidant agents include, but are not limited to, colloidal silica, calcium silicate, magnesium silicate, silicon hydrogel, cornstarch, talc, combinations thereof and other such materials known to those of ordinary skill in the art.

Lubricant agents include, but are not limited to, calcium stearate, magnesium stearate, mineral oil, stearic acid, zinc stearate, glycerin behenate, hydrogenated vegetable oil, sodium stearyl fumarate and myristic Acid suitable combinations thereof and other such materials known to those of ordinary skill in the art.

Coating redimix is selected from Opadry Pink all such materials known to those of ordinary skill in the art.

In certain embodiments, compounds of formula (Ia) or its pharmaceutically acceptable salts, for administration to a subject at a dose in the range of 1 mg to 500 mg. In certain embodiments, the compound of formula (Ia) or a pharmaceutically acceptable salts for administration to a subject to provide the compound of formula (Ia) or a pharmaceutically acceptable salts at a dose of 1 mg to 150 mg. In certain embodiments, the compound of formula (Ia) or a pharmaceutically acceptable salts for administration to a subject to provide the compound of formula (Ia) or a pharmaceutically acceptable salts at a dose of 25 mg, 50 mg and 100 mg to the subject.

In a further embodiment, the compound of formula (Ia) or its pharmaceutically acceptable salts is administered to a subject in an amount of about 1 mg to about 500 mg, about 50 mg to about 450 mg, about 100 mg to about 400 mg, about 150 mg to about 350 mg, about 200 mg to about 300 mg, about 1 mg to about 50 mg, about 1 mg to about 25 mg to the subject.

In a further embodiment, Iptacopan is administered to a subject in an amount of about 1 mg to about 500 mg, about 50 mg to about 450 mg, about 100 mg to about 400 mg, about 150 mg to about 350 mg, about 200 mg to about 300 mg. In a certain embodiment, Iptacopan is administered in an amount of 200 mg.

In certain embodiments, compounds of formula (Ia) or its pharmaceutically acceptable salts can be further characterized according to the dose of compound administered to a subject, where subject is animal or human.

In a further embodiment, the present invention provides effective amount of compound of formula (Ia) or its pharmaceutically acceptable salt is administered to subject by oral, parenteral, intravenous or intramuscular route of administration.

### Pharmaceutical Composition of Compound of Formula (Ia) or its Pharmaceutically Acceptable Salts and Suitable Angiotensin II Receptor Antagonist for Use in Treating Glomerular Diseases.

In an embodiment, present invention provides a pharmaceutical composition comprising compound of formula (Ia) or its pharmaceutically acceptable salts and suitable Angiotensin II receptor antagonist optionally with one or more pharmaceutically acceptable excipients for use in treating glomerular diseases.

The glomerular disease includes Nephrotic Syndrome, Focal Segmental Glomerulosclerosis (FSGS), Minimal Change Disease (MCD), Membranous Nephropathy (MN), IgA Nephropathy (IgAN), C3 Glomerulopathy (MPGN3), Diabetic nephropathy, Lupus nephritis and other condition associated with Glomerular Diseases.

Pharmaceutically acceptable salts of the compound of formula (Ia) is selected from metal salt, amine base salt and amino acid salt.

Wherein metal salt is selected from calcium, sodium, potassium, lithium, barium, strontium, magnesium, cesium, copper, cobalt, iron, manganese, lead, aluminum, cadmium, silver, zinc, ammonium and the like; wherein amine base salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane-1,6-diamine, 2-(2-aminoethoxy) ethanamine, N-methylmorpholine, and N-ethylmorpholine; wherein amino acid salt is selected from alanine, lysine, arginine, histidine, threonine, proline, glutamine and glycine. The Angiotensin II receptor antagonist is Fimasartan, Azilsartan, Candesartan, Eprosartan, Losartan, Olmesartan, Telmisartan and Valsartan.

The pharmaceutically acceptable excipients are selected at least one from diluent, binders, disintegrating agents, lubricating agents, glidant agent, sweetener, pH modifier, suspending agent or viscosity modifying agent, flavouring agent and optionally coating redimix.

Diluents include, but are not limited to lactose monohydrate, lactose, microcrystalline cellulose, polymethacrylates selected from Eudragit, potassium chloride, sulfobutylether b-cyclodextrin, sodium chloride, spray dried lactose, and preferably sulfobutyl ether b-cyclodextrin combinations thereof and other such materials known to those of ordinary skill in the art.

Binders include, but are not limited to hypromellose 3 Cps, carbomers selected from carbopol, gellan, gum Arabic, hydrogenated vegetable oil, polymethacrylates selected from Eudragit, xanthan, lactose and Zein combinations thereof and other such materials known to those of ordinary skill in the art.

Disintegrating agents include, but are not limited to, croscarmellose Sodium, bicarbonate salt, chitin, gellan gum, polacrillin potassium and docusate Sodium combinations thereof and other such materials known to those of ordinary skill in the art.

Glidant agents include, but are not limited to, colloidal silica, calcium silicate, magnesium silicate, silicon hydrogel, corn starch, talc, combinations thereof and other such materials known to those of ordinary skill in the art.

Lubricant agents include, but are not limited to, calcium stearate, magnesium stearate, mineral oil, stearic acid, zinc stearate, glycerin behenate, hydrogenated vegetable oil, sodium stearyl fumarate and myristic Acid suitable combinations thereof and other such materials known to those of ordinary skill in the art.

Coating redimix is selected from Opadry Pink all such materials known to those of ordinary skill in the art.

In certain embodiments, compounds of formula (Ia) or its pharmaceutically acceptable salts, for administration to a subject at a dose in the range of 1 mg to 500 mg. In certain embodiments, the compound of formula (Ia) or a pharmaceutically acceptable salts for administration to a subject to provide the compound of formula (Ia) or a pharmaceutically acceptable salts at a dose of 1 mg to 150 mg. In certain embodiments, the compound of formula (Ia) or a pharmaceutically acceptable salts for administration to a subject to provide the compound of formula (Ia) or a pharmaceutically acceptable salts at a dose of 25 mg, 50 mg and 100 mg to the subject.

In a further embodiment, the compound of formula (Ia) or its pharmaceutically acceptable salts is administered to a subject in an amount of about 1 mg to about 500 mg, about 50 mg to about 450 mg, about 100 mg to about 400 mg, about 150 mg to about 350 mg, about 200 mg to about 300 mg, about 1 mg to about 50 mg, about 1 mg to about 25 mg to the subject.

In a further embodiment, Fimasartan is administered to a subject in an amount of about 1 mg to about 500 mg, about 50 mg to about 450 mg, about 100 mg to about 400 mg, about 150 mg to about 350 mg, about 200 mg to about 300 mg. In a further embodiment, Azilsartan is administered to a subject in an amount of about 1 mg to about 500 mg. In a further embodiment, Candesartan is administered to a subject in an amount of about 1 mg to about 200 mg. In a further embodiment, Eprosartan is administered to a subject in an amount of about 1 mg to about 1000 mg. In a further embodiment, Losartan is administered to a subject in an amount of about 1 mg to about 500 mg. In a further embodiment, Olmesartan is administered to a subject in an amount of about 1 mg to about 200 mg. In a further embodiment, Telmisartan is administered to a subject in an amount of about 1 mg to about 500 mg. In a further embodiment, Valsartan is administered to a subject in an amount of about 1 mg to about 500 mg.

In a certain embodiment, Fimasartan is administered to a subject in an amount of about 60 mg and 120 mg. In a further embodiment, Azilsartan is administered to a subject in an amount of about 40 mg and 80 mg. In a further embodiment, Candesartan is administered to a subject in an amount of about 4 mg, 8 mg, 16 mg and 32 mg. In a further embodiment, Eprosartan is administered to a subject in an amount of about 400 mg and 600 mg. In a further embodiment, Losartan is administered to a subject in an amount of about 25 mg, 50 mg and 100 mg. In a further embodiment, Olmesartan is administered to a subject in an amount of about 5 mg, 20 mg and 40 mg. In a further embodiment, Telmisartan is administered to a subject in an amount of about 20 mg, 40 mg and 80 mg. In a further embodiment, Valsartan is administered to a subject in an amount of about 40 mg, 80 mg, 160 mg and 320 mg.

In certain embodiments, compounds of formula (Ia) or its pharmaceutically acceptable salts can be further characterized according to the dose of compound administered to a subject, where subject is animal or human.

In a further embodiment, the present invention provides effective amount of compound of formula (Ia) or its pharmaceutically acceptable salt is administered to subject by oral, parenteral, intravenous or intramuscular route of administration.

### Use of Compound of Formula (Ia) or its Pharmaceutically Acceptable Salts for the Treatment of Glomerular Diseases

In an embodiment, present invention provides use of compound of formula (I) or its pharmaceutically acceptable salts for the treatment of glomerular diseases.

The glomerular disease includes Nephrotic Syndrome, Focal Segmental Glomerulosclerosis (FSGS), Minimal Change Disease (MCD), Membranous Nephropathy (MN), IgA Nephropathy (IgAN), C3 Glomerulopathy (MPGN3), Diabetic nephropathy, Lupus nephritis and other condition associated with Glomerular Diseases.

Pharmaceutically acceptable salts of the compound of formula (Ia) is selected from metal salt, amine base salt and amino acid salt.

Wherein metal salt is selected from calcium, sodium, potassium, lithium, barium, strontium, magnesium, cesium, copper, cobalt, iron, manganese, lead, aluminum, cadmium, silver, zinc, ammonium and the like; wherein amine base salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane-1,6-diamine, 2-(2-aminoethoxy) ethanamine, N-methylmorpholine, and N-ethylmorpholine; wherein amino acid salt is selected from alanine, lysine, arginine, histidine, threonine, proline, glutamine and glycine. In certain embodiments, compounds of formula (Ia) or its pharmaceutically acceptable salts, for administration to a subject at a dose in the range of 1 mg to 500 mg. In certain embodiments, the compound of formula (Ia) or a pharmaceutically acceptable salts for administration to a subject to provide the compound of formula (Ia) or a pharmaceutically acceptable salts at a dose of 1 mg to 150 mg. In certain embodiments, the compound of formula (Ia) or a pharmaceutically acceptable salts for administration to a subject to provide the compound of formula (Ia) or a pharmaceutically acceptable salts at a dose of 25 mg, 50 mg and 100 mg to the subject.

In a further embodiment, the compound of formula (Ia) or its pharmaceutically acceptable salts is administered to a subject in an amount of about 1 mg to about 500 mg, about 50 mg to about 450 mg, about 100 mg to about 400 mg, about 150 mg to about 350 mg, about 200 mg to about 300 mg, about 1 mg to about 50 mg, about 1 mg to about 25 mg to the subject.

In certain embodiments, compounds of formula (Ia) or its pharmaceutically acceptable salts can be further characterized according to the dose of compound administered to a subject, where subject is animal or human.

In a further embodiment, the present invention provides effective amount of compound of formula (Ia) or its pharmaceutically acceptable salt is administered to subject by oral, parenteral, intravenous or intramuscular route of administration.

Use of Compound of Formula (Ia) or its Pharmaceutically Acceptable Salts in Combination with Suitable Factor B for the Treatment of Glomerular Diseases.

In an embodiment, present invention provides use of combination of compound of formula (I) or its pharmaceutically acceptable salts with suitable Factor B inhibitors for the treatment of glomerular diseases.

The glomerular disease includes Nephrotic Syndrome, Focal Segmental Glomerulosclerosis (FSGS), Minimal Change Disease (MCD), Membranous Nephropathy (MN), IgA Nephropathy (IgAN), C3 Glomerulopathy (MPGN3), Diabetic nephropathy, Lupus nephritis and other condition associated with Glomerular Diseases.

Pharmaceutically acceptable salts of the compound of formula (Ia) is selected from metal salt, amine base salt and amino acid salt.

Wherein metal salt is selected from calcium, sodium, potassium, lithium, barium, strontium, magnesium, cesium, copper, cobalt, iron, manganese, lead, aluminum, cadmium, silver, zinc, ammonium and the like; wherein amine base salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane-1,6-diamine, 2-(2-aminoethoxy) ethanamine, N-methylmorpholine, and N-ethylmorpholine; wherein amino acid salt is selected from alanine, lysine, arginine, histidine, threonine, proline, glutamine and glycine. Factor B inhibitor use in combination with compound of formula (Ia) or its pharmaceutically acceptable salts is Iptacopan for the treatment of glomerular diseases.

In certain embodiments, compounds of formula (Ia) or its pharmaceutically acceptable salts, for administration to a subject at a dose in the range of 1 mg to 500 mg. In certain embodiments, the compound of formula (Ia) or a pharmaceutically acceptable salts for administration to a subject to provide the compound of formula (Ia) or a pharmaceutically acceptable salts at a dose of 1 mg to 150 mg. In certain embodiments, the compound of formula (Ia) or a pharmaceutically acceptable salts for administration to a subject to provide the compound of formula (Ia) or a pharmaceutically acceptable salts at a dose of 25 mg, 50 mg and 100 mg to the subject.

In a further embodiment, the compound of formula (Ia) or its pharmaceutically acceptable salts is administered to a subject in an amount of about 1 mg to about 500 mg, about 50 mg to about 450 mg, about 100 mg to about 400 mg, about 150 mg to about 350 mg, about 200 mg to about 300 mg, about 1 mg to about 50 mg, about 1 mg to about 25 mg to the subject.

In a further embodiment, Iptacopan is administered to a subject in an amount of about 1 mg to about 500 mg, about 50 mg to about 450 mg, about 100 mg to about 400 mg, about 150 mg to about 350 mg, about 200 mg to about 300 mg. In a certain embodiment, Iptacopan is administered in an amount of 200 mg.

In certain embodiments, compounds of formula (Ia) or its pharmaceutically acceptable salts can be further characterized according to the dose of compound administered to a subject, where subject is animal or human.

In a further embodiment, the present invention provides effective amount of compound of formula (Ia) or its pharmaceutically acceptable salt is administered to subject by oral, parenteral, intravenous or intramuscular route of administration.

Use of Compound of Formula (Ia) or its Pharmaceutically Acceptable Salts in Combination with Suitable Angiotensin II Receptor Antagonist for the Treatment of Glomerular Diseases.

In an embodiment, present invention provides use of combination of compound of formula (I) or its pharmaceutically acceptable salts with suitable Angiotensin II receptor antagonist for the treatment of glomerular diseases.

The glomerular disease includes Nephrotic Syndrome, Focal Segmental Glomerulosclerosis (FSGS), Minimal Change Disease (MCD), Membranous Nephropathy (MN), IgA Nephropathy (IgAN), C3 Glomerulopathy (MPGN3), Diabetic nephropathy, Lupus nephritis and other condition associated with Glomerular Diseases.

Pharmaceutically acceptable salts of the compound of formula (Ia) is selected from metal salt, amine base salt and amino acid salt.

Wherein metal salt is selected from calcium, sodium, potassium, lithium, barium, strontium, magnesium, cesium, copper, cobalt, iron, manganese, lead, aluminum, cadmium, silver, zinc, ammonium and the like; wherein amine base salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane-1,6-diamine, 2-(2-aminoethoxy) ethanamine, N-methylmorpholine, and N-ethylmorpholine; wherein amino acid salt is selected from alanine, lysine, arginine, histidine, threonine, proline, glutamine and glycine.

Angiotensin II receptor antagonist use in combination with compound of formula (Ia) or its pharmaceutically acceptable salts is Fimasartan, Azilsartan, Candesartan, Eprosartan, Losartan, Olmesartan, Telmisartan and Valsartan for the treatment of glomerular diseases.

In certain embodiments, compounds of formula (Ia) or its pharmaceutically acceptable salts, for administration to a subject at a dose in the range of 1 mg to 500 mg. In certain embodiments, the compound of formula (Ia) or a pharmaceutically acceptable salts for administration to a subject to provide the compound of formula (Ia) or a pharmaceutically acceptable salts at a dose of 1 mg to 150 mg. In certain embodiments, the compound of formula (Ia) or a pharmaceutically acceptable salts for administration to a subject to provide the compound of formula (Ia) or a pharmaceutically acceptable salts at a dose of 25 mg, 50 mg and 100 mg to the subject.

In a further embodiment, the compound of formula (Ia) or its pharmaceutically acceptable salts is administered to a subject in an amount of about 1 mg to about 500 mg, about 50 mg to about 450 mg, about 100 mg to about 400 mg, about 150 mg to about 350 mg, about 200 mg to about 300 mg, about 1 mg to about 50 mg, about 1 mg to about 25 mg to the subject.

In a further embodiment, Fimasartan is administered to a subject in an amount of about 1 mg to about 500 mg, about 50 mg to about 450 mg, about 100 mg to about 400 mg, about 150 mg to about 350 mg, about 200 mg to about 300 mg. In a further embodiment, Azilsartan is administered to a subject in an amount of about 1 mg to about 500 mg. In a further embodiment, Candesartan is administered to a subject in an amount of about 1 mg to about 200 mg. In a further embodiment, Eprosartan is administered to a subject in an amount of about 1 mg to about 1000 mg. In a further embodiment, Losartan is administered to a subject in an amount of about 1 mg to about 500 mg. In a further embodiment, Olmesartan is administered to a subject in an amount of about 1 mg to about 200 mg. In a further embodiment, Telmisartan is administered to a subject in an amount of about 1 mg to about 500 mg. In a further embodiment, Valsartan is administered to a subject in an amount of about 1 mg to about 500 mg.

In a certain embodiment, Fimasartan is administered to a subject in an amount of about 60 mg and 120 mg. In a further embodiment, Azilsartan is administered to a subject in an amount of about 40 mg and 80 mg. In a further embodiment, Candesartan is administered to a subject in an amount of about 4 mg, 8 mg, 16 mg and 32 mg. In a further embodiment, Eprosartan is administered to a subject in an amount of about 400 mg and 600 mg. In a further embodiment, Losartan is administered to a subject in an amount of about 25 mg, 50 mg and 100 mg. In a further embodiment, Olmesartan is administered to a subject in an amount of about 5 mg, 20 mg and 40 mg. In a further embodiment, Telmisartan is administered to a subject in an amount of about 20 mg, 40 mg and 80 mg. In a further embodiment, Valsartan is administered to a subject in an amount of about 40 mg, 80 mg, 160 mg and 320 mg.

In certain embodiments, compounds of formula (Ia) or its pharmaceutically acceptable salts can be further characterized according to the dose of compound administered to a subject, where subject is animal or human.

In a further embodiment, the present invention provides effective amount of compound of formula (Ia) or its pharmaceutically acceptable salt is administered to subject by oral, parenteral, intravenous or intramuscular route of administration.

### Reduction in the Amount Urine Protein

In an embodiment, compound of formula (Ia) or its pharmaceutically acceptable salts alone or in combination with suitable other inhibitors when administered in subject according to a reduction in the amount of urine protein.

For example, in certain embodiments, there is a reduction in the amount of urine protein.

In certain embodiments, there is at least a 5% reduction in the amount of urine protein. In certain embodiments, there is at least a 10% reduction in the amount of urine protein. In certain embodiments, there is at least a 15% reduction in the amount of urine protein. In certain embodiments, there is at least a 20% reduction in the amount of urine protein. In certain embodiments, there is at least a 25% reduction in the amount of urine protein. In certain embodiments, there is at least a 30% reduction in the amount of urine protein. In certain embodiments, there is at least a 35% reduction in the amount of urine protein. In certain embodiments, there is at least a 40% reduction in the amount of urine protein. In certain embodiments, there is at least a 50% reduction in the amount of urine protein. In certain embodiments, there is at least a 60% reduction in the amount of urine protein. In certain embodiments, there is at least a 70% reduction in the amount of urine protein. In certain embodiments, there is at least an 80% reduction in the amount of urine protein. In certain embodiments, there is at least a 90% reduction in the amount of urine protein.

### Dosing Schedule

The method may be further characterized according to a dosing schedule by which the compound of formula (Ia) or its pharmaceutically acceptable salts alone or in combination, pharmaceutical composition of compound of formula (Ia) or its pharmaceutically acceptable salts alone or in combination is administered.

In certain embodiments, compound is administered to the subject once a daily, twice a daily and thrice a daily. In another embodiments, compound is administered to the subject for at least 1 week. In another embodiments, compound is administered to the subject for at least 2 week. In another embodiments, compound is administered to the subject for at least 3 week. In another embodiments, compound is administered to the subject for at least 4 week. In another embodiments, compound is administered to the subject for at least 6 week. In another embodiments, compound is administered to the subject for at least 8 week. In another embodiments, compound is administered to the subject for at least 10 week. In another embodiments, compound is administered to the subject for at least 12 week. In another embodiments, compound is administered to the subject for at least 14 week. In another embodiments, compound is administered to the subject for at least 16 week.

In an embodiment, the present invention provides a method of treating glomerular disease in a patient with diabetes, comprising administering an effective amount of compound of formula (Ia) or its pharmaceutically acceptable salts or combination of compound of formula (Ia) or its pharmaceutically acceptable salts with suitable Factor B inhibitors or Angiotensin II receptor antagonist.

The scope of pharmaceutically acceptable salts, dosage form, suitable Factor B inhibitors, suitable Angiotensin II receptor antagonist, glomerular disease as define anywhere in the specification.

In another embodiment the present invention provides compound of formula (Ia) or its pharmaceutically acceptable salt in combination of other prolyl hydroxylase inhibitors such as Roxadustat, Vadadustat, Molidustat, Daprodustat and the like.

Compound of formula (Ia) is prepared as per method disclosed in WO 2014102818.

Iptacopan is prepared as per method disclosed in WO2015009616.

Fimasartan is prepared as per method disclose in WO199955681.

### EXAMPLES

The effect of compounds of formula (Ia) and its combination thereof in animal models for glomerular disease is shown herein below.

### Animal Models of Glomerular Disease

### Example 1: Effect of Compound of Formula (Ia) and Combination Thereof on LPS-Induced Nephritis in C57 Mice

Single dose of lipopolysachharide (LPS)-induced acute nephritis was generated in C57 mice and the effect of compound of formula (Ia) and combination as shown below.

Male C57 mice were treated with vehicle/compound of formula (Ia) (15 mg/kg) and combination of compound of formula (Ia) (15 mg/kg) and Iptacopan (20 mg/kg, twice a day) by oral route. These mice were bled after 24 h of LPS treatment and serum was obtained. The creatinine and urea levels were measured in the serum.

| Sr. No. | Compound | Serum creatinine | Serum urea |
|---|---|---|---|
| 1 | Formula (Ia) | 34.1 ± 5.2% | 44.9 ± 5.2% |
| 2 | Combination of Formula | 55.6 ± 4.8% | 65.6 ± 3.2% |
| | (Ia) and Iptacopan | | |

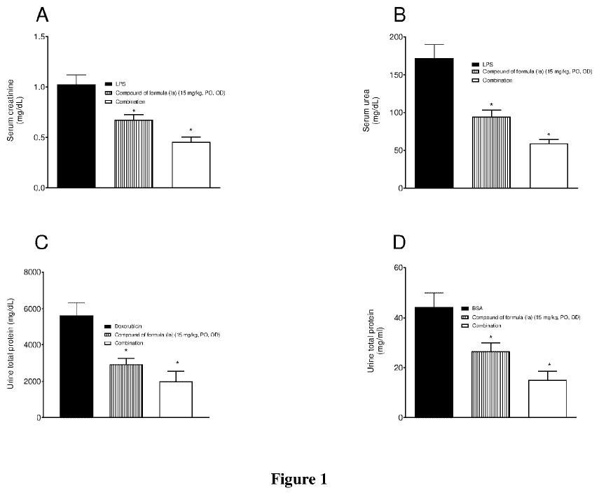

Acute treatment with compound of formula (Ia) reduced serum creatinine by 34.1±5.2 against LPS treated mice while combination of compound of formula (Ia) and Iptacopan reduced serum creatinine by 55.6±4.8% (FIG. 1A). Serum urea was decreased by compound of formula (Ia) by 44.9 +5.2 while combination of Iptacopan and compound of formula (Ia) reduced it by 65.6±3.2% when compared with LPS treated mice (FIG. 1B).

### Example 2: Effect of Compound of Formula (Ia) on 5/6 Nephrectomy Induced Renal Dysfunction in Male SD Rats

Male SD rats will be operated for 5/6 nephrectomy to induce the focal segmental glomerulosclerosis. After a week of recovery, animals were treated with compound of formula (Ia). The randomized rats were treated with compound of formula (Ia) (15 mg/kg) for four weeks. At the end of treatment serum creatinine, serum urea and urine microalbumin was measured.

| | | | Serum | Serum |
|---|---|---|---|---|
| Sr. No. | Compound | Microalbumin | creatinine | urea |
| 1 | Formula (Ia) | 35.9 ± 12.0% | 14.9 ± 3.2% | 15.9 ± 8.8% |

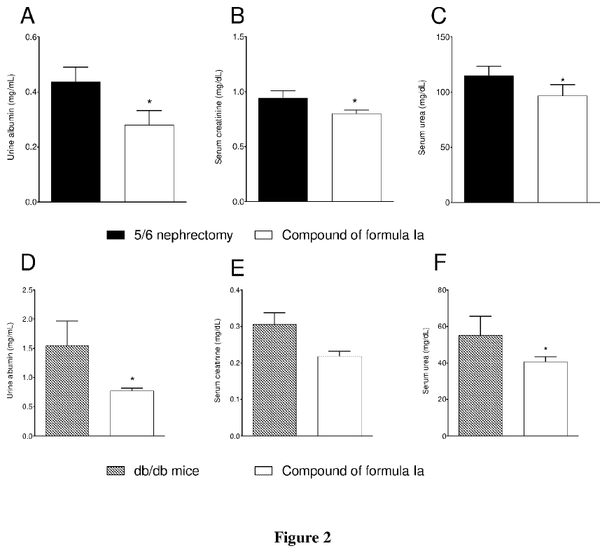

Compound of formula (Ia) treatment reduced urinary excretion of microalbumin by 35.9±12.0 when compared against diabetic mice (FIG. 2A). It also reduced serum creatinine and urea by 14.9±3.2 and 15.9±8.8%, respectively (FIG. 2B-C).

### Example 3: Effect of Compound of Formula (Ia) or Combination Thereof on Doxorubicin-Induced Glomerulosclerosis in Male Balb/c Mice

Male Balb/c mice were treated with doxorubicin (10 mg/kg, IV). After 5 weeks of treatment, mice were randomized based on protein excretion urine into four groups: Doxorubicin, compound of formula (Ia) (15 mg/kg) and combination of compound of formula (Ia) (15 mg/kg) and Fimasartan (15 mg/kg) by oral route. The treatment continued for two weeks. These mice kept in metabolic cage and 24 h urine protein excretion was measured.

| Sr. No. | Compound | Urine total protein |
|---|---|---|
| 1 | Formula (Ia) | 47.8 ± 5.8% |
| 2 | Combination of Formula (Ia) and | 64.4 ± 10.1% |
| | Fimasartan | |

After two weeks of treatment compound of formula (Ia) reduced excretion of total protein in urine by 47.8±5.8 against doxorubicin treated animals. Combination of Fimasartan and compound of formula (Ia) reduced proteinuria by 64.4±10.1% against doxorubicin treatment FIG. 1C.

### Example 4: Effect of Compound of Formula (Ia) or Combination Thereof on Cationic Bovine Serum Slbumin (cBSA) Induced Nephritis in Male C57 Mice

Male C57 mice were treated with bovine serum albumin (BSA) at 2, 4, 6, 8 and 10 mg/kg on day 1, 2, 3, 4, and 5 days by intraperitoneal route, respectively. The BSA at 10 mg/kg was treated for next 5 days. On the same day, mice were treated with either vehicle/compound of formula (Ia) (15 mg/kg) and combination of compound of formula (Ia) (15 mg/kg) and Iptacopan (20 mg/kg, BID) by oral route and continued for 10 days. On 11th day mice kept in metabolic cage and 24 h urine protein excretion was measured.

| Sr. No. | Compound | Urine total protein |
|---|---|---|
| 1 | Formula (Ia) | 40.1 ± 7.6% |
| 2 | Combination of Formula (Ia) and | 65.7 ± 7.8% |
| | Iptacopan | |

Compound of formula (Ia) treatment reduced excretion of total protein by 40.1±7.6% against BSA treated mice. Combination of Iptacopan and compound of formula (Ia) treatment reduced proteinuria by 65.7±7.8% (FIG. 1D).

### Example 5: Diabetes Induced Renal Dysfunction and Effect of Formula I(a)

For inducing diabetes-induced renal dysfunction, one kidney was removed from male db/db mice and they were treated with compound of formula (Ia) and combination for 8 weeks. At the end of treatment, renal dysfunction was estimated. Histological change in renal disease was also accessed with biochemical changes in serum and urine.

Male db/db mice were randomized and were treated with compound of formula (Ia) (15 mg/kg) for eight weeks. At the end of treatment serum creatinine, serum urea and urine microalbumin was measured.

| | | Urine | Serum | Serum |
|---|---|---|---|---|
| Sr. No. | Compound | microalbumin | creatinine | urea |
| 1 | Formula (Ia) | 50.2 ± 3.2% | 28.4 ± 4.4% | 25.8 ± 4.8% |

Compound of formula (Ia) treatment reduced urinary excretion of microalbumin by 50.2±3.2 when compared against diabetic mice (FIG. 2D). It also reduced serum creatinine and urea by 28.4±4.4 and 25.8±4.8%, respectively (FIG. 2E-F).

Compound of formula (Ia) alone or in combination with Iptacopan or Fimasartan showed synergistic effect as described and elucidated in above table.

### REFERENCES

1. Wang B, Li Z L, Zhang Y L, Wen Y, Gao Y M, Liu B C. Hypoxia and chronic kidney disease. eBioMedicine [Internet]. 2022 Mar. 1 [cited 2022 Jun. 17]; 77:103942. Available from: http://www.thelancet.com/article/S2352396422001268/fulltext
2. Fu Q, Colgan S P, Shelley C S. Hypoxia: The Force that Drives Chronic Kidney Disease. Clin Med Res [Internet]. 2016 Mar. 1 [cited 2022 Jun. 17]; 14(1):15. Available from: /pmc/articles/PMC4851450/3.
3. Neusser M A, Lindenmeyer M T, Moll A G, Segerer S, Edenhofer I, Sen K, et al. Human Nephrosclerosis Triggers a Hypoxia-Related Glomerulopathy. Am J Pathol [Internet]. 2010 [cited 2022 Jun. 17]; 176(2):594. Available from: /pmc/articles/PMC2808068/4.
4. Joharapurkar A A, Pandya V B, Patel V J, Desai R C, Jain M R. Prolyl Hydroxylase Inhibitors: A Breakthrough in the Therapy of Anemia Associated with Chronic Diseases. J Med Chem [Internet]. 2018 Aug. 23 [cited 2022 Jun. 17]; 61(16):6964-82. Available from: https://pubs.acs.org/doi/abs/10.1021/acs.jmedchem.7b01686
5. Nordquist L, Friederich-Persson M, Fasching A, Liss P, Shoji K, Nangaku M, et al. Activation of hypoxia-inducible factors prevents diabetic nephropathy. J Am Soc Nephrol [Internet]. 2015 Feb. 1 [cited 2022 Jun. 17]; 26(2):328-38. Available from: https://jasn.asnjournals.org/content/26/2/328
6. Hill P, Shukla D, Tran M G B, Aragones J, Cook H T, Carmeliet P, et al. Inhibition of Hypoxia Inducible Factor Hydroxylases Protects Against Renal Ischemia-Reperfusion Injury. J Am Soc Nephrol [Internet]. 2008 Jan. 1 [cited 2022 Jun. 17]; 19(1):39-46. Available from: https://jasn.asnjournals.org/content/19/1/39
7. Taylor C T, Doherty G, Fallon P G, Cummins E P. Hypoxia-dependent regulation of inflammatory pathways in immune cells. J Clin Invest [Internet]. 2016 Oct. 10 [cited 2022 Jun. 17]; 126(10):3716. Available from: /pmc/articles/PMC5096820/8.
8. Imtiyaz H Z, Simon MC. Hypoxia-inducible factors as essential regulators of inflammation. Curr Top Microbiol Immunol [Internet]. 2010 [cited 2022 Jun. 17]; 345:105. Available from: /pmc/articles/PMC3144567/9.
9. Scholz C C, Cavadas M A S, Tambuwala M M, Hams E, Rodriguez J, Von Kriegsheim A, et al. Regulation of IL-1β-induced NF-κB by hydroxylases links key hypoxic and inflammatory signaling pathways. Proc Natl Acad Sci USA [Internet]. 2013 Nov. 12 [cited 2022 Jun. 17]; 110(46):18490-5. Available from:

1. A method of treating glomerular disease in a subject, comprising administering an effective amount of compound of formula (Ia) or its pharmaceutically acceptable salts, wherein formula (Ia) is represented by:

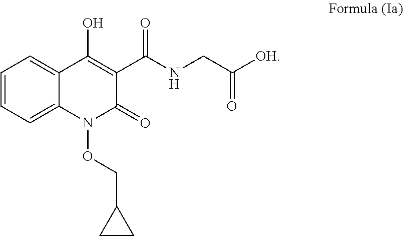 [SMILES: O=C(O)CNC(=O)c1c(O)c2ccccc2n(OCC2CC2)c1=O]

2. The method of claim 1, wherein the glomerular disease is selected from Nephrotic Syndrome, Focal Segmental Glomerulosclerosis (FSGS), Minimal Change Disease (MCD), Membranous Nephropathy (MN), IgA Nephropathy (IgAN), C3 Glomerulopathy (MPGN), Diabetic nephropathy and Lupus nephritis.

3. The method of claim 1, wherein pharmaceutically acceptable salts of the compound of formula (Ia) is selected from metal salt, amine base salt and amino acids salt.

4. The method of claim 3, wherein the metal salt is selected from calcium, sodium, potassium, lithium, barium, strontium, magnesium, cesium, copper, cobalt, iron, manganese, lead, aluminum, cadmium, silver, zinc, ammonium and the like;
wherein amine base salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N-N-diethylethanolamine, deanol, hydrabamine, betaine, adamantanamine, L-adamantanmethylamine, tritylamine, glucamine, N-methyl pyrrolidine, urea, procaine, metformin, hexane-1,6-diamine, 2-(2-aminoethoxy) ethanamine, N-methylmorpholine, and N-ethylmorpholine; wherein amino acid salt is selected from alanine, lysine, arginine, histidine, threonine, proline, glutamine and glycine.

5. The method of claim 1, wherein the compound of formula (Ia) or its pharmaceutically acceptable salt is administered to a subject in amount of 1 mg to 500 mg, 50 mg to 450 mg, 100 mg to 400 mg, 150 mg to 350 mg, 200 mg to 300 mg, 1 mg to 50 mg, 1 mg to 25 mg to the subject.

6. The method of claim 1, wherein the compound of formula (Ia) or its pharmaceutically acceptable salt is administered to a subject at a dose of 1 mg to 150 mg.

7. The method of claim 1, wherein the compound of formula (Ia) or its pharmaceutically acceptable salt is administered to a subject at a dose of 25 mg, 50 mg and 100 mg.

8. The method of claim 1, wherein the compound of formula (Ia) or its pharmaceutically acceptable salt is administered to a subject, where subject is animal or human.

9. The method of claim 1, wherein the compound of formula (Ia) or its pharmaceutically acceptable salt is administered to a subject by oral, parenteral, intravenous or intramuscular route of administration.

10-44. (canceled)

45. A combination of compound of formula (Ia) or its pharmaceutically acceptable salts with suitable second therapeutic agent, wherein suitable second therapeutic agent selected from suitable Factor B inhibitors or suitable Angiotensin II receptor antagonist or suitable Factor D inhibitors or suitable C3 inhibitors or suitable C5 inhibitors or suitable C6 inhibitors or suitable Lectin pathway inhibitors or suitable Properdin inhibitors or suitable C9 antibody and multitarget complement inhibitor.

46. The combination as claimed in claim 45, wherein suitable second therapeutic agent is suitable Factor B inhibitors or suitable Angiotensin II receptor antagonist.

47. The combination as claimed in claim 46, wherein suitable Factor B inhibitors is Iptacopan and suitable Angiotensin II receptor antagonist is Fimasartan, Azilsartan, Candesartan, Eprosartan, Losartan, Olmesartan, Telmisartan and Valsartan; wherein Factor B inhibitors is administered to a subject in an amount of about 1 mg to 500 mg and Angiotensin II receptor antagonist is administered to a subject in an amount of about 1 mg to 500 mg.

48-91. (canceled)

92. A method of treating glomerular disease in a subject as claimed in claim 1 wherein subject is a patient with diabetes.

93-104. (canceled)

105. A method of treating glomerular disease in a subject, by administering a combination of compound of formula (Ia) or its pharmaceutically acceptable salts with suitable second therapeutic agent as claimed in claim 45, wherein suitable second therapeutic agent selected from suitable Factor B inhibitors or suitable Angiotensin II receptor antagonist or suitable Factor D inhibitors or suitable C3 inhibitors or suitable C5 inhibitors or suitable C6 inhibitors or suitable Lectin pathway inhibitors or suitable Properdin inhibitors or suitable C9 antibody and multitarget complement inhibitor.
